(19) United States
(12) Patent Application Publication
(10) Pub. No.: US 2005/0084503 A1
(43) Pub. Date: Apr. 21, 2005

(54) Continuous cell line for the production of vaccines

(76) Inventors: Nikolaus Osterrieder, Benningen (DE); Daniel Schumacher, Ithaca, NY (US)

Correspondence Address: TRASK BRITT, P.O. BOX 2550, SALT LAKE CITY, UT, 84110, US

(21) Appl. No.: 10/912,535

(22) Filed: Aug. 4, 2004

Related U.S. Application Data
(63) Continuation of application No. PCT/EP03/01213, filed on Feb. 5, 2003.

(30) Foreign Application Priority Data
Feb. 6, 2002 (EP) 02075480.0

(51) Int. Cl. A61K 39/245; C12N 7/00

(52) U.S. Cl. 424/229.1; 435/235.1

(57) ABSTRACT

Provided is a method for producing a continuous cell line capable of supporting the growth of a Marek's disease virus (MDV) strain comprising infecting or transfecting a cell with a vector comprising a nucleic acid or fragment thereof of an MDV strain and culturing the cell or a progeny thereof under conditions suitable for the expression of the nucleic acid and propagation of the cell or progeny. The nucleic acid may comprise an MDV glycoprotein gE gene or functional fragment. Also provided is a method of generating, isolating, and/or maintaining a hypervirulent, very virulent, very virulent plus, virulent, and/or avirulent MDV strain comprising infecting a described cell with the MDV strain or strains, and culturing the cell under conditions suitable for the propagation of the cell and the generation, maintenance and isolation of the MDV strain or strains. Also provided is a method to prepare a vaccine capable of inducing protection against disease, preferably an MDV associated disease in an avian, including culturing a continuous cell according to the invention and harvesting cell culture components therefrom.

### CROSS-REFERENCE TO RELATED APPLICATION

This application is a continuation of PCT International Patent Application No. PCT/EP03/01213, filed on Feb. 5, 2003, designating the United States of America, and published, in English, as PCT International Publication No. WO 03/066093 A1 on Aug. 14, 2004, the contents of the entirety of which is incorporated by this reference.

### TECHNICAL FIELD

The invention relates to the field of immunology and biotechnology. In particular, it relates to the production of a cell line suited for the production of a vaccine against animal diseases, more in particular avian diseases, caused by herpes viruses, more in particular alpha herpes viruses. It also relates to the production of a cell line suited for the isolation of existing and newly emerging alpha herpes viruses, such as for example avirulent, virulent, and very virulent (or hypervirulent) Marek's disease virus strains.

### BACKGROUND

Marek's disease (MD) is a devastating disorder that affects chickens worldwide. It is caused by Marek's disease virus (MDV), an alpha herpes virus, and characterized by T cell lymphomas, polyneuritis, general immunosuppression, and rarely atherosclerosis⁵. Three MDV serotypes have been identified, out of which only serotype 1 (MDV-1) is pathogenic, whereas MDV serotype 2 (MDV-2) and serotype 3 (herpes virus of turkeys, HVT) do not cause MD. MDV infection results in the establishment of latent infection which is common in alpha-herpesvirinae, however, MDV has unique properties when compared to other members of this virus subfamily because it can cause tumors and integrate into the host cell genome^{11,12}.

MD can be controlled by immunization, but despite vaccination more and more pathogenic MDV strains have evolved⁴. These strains have been classified as so-called virulent (v), very virulent (vv) or very virulent plus (vv+) strains^{28,35}. Vaccination using HVT was capable of protecting chickens against infections with vMDV, but failed to protect against newly emerging vvMDV in the late 1970's³⁸. Vaccines containing MDV-2 strains or combinations of HVT and MDV-2 were used successfully in the 1980's^{6,34,37,39}, but within few years novel vv+ MDV-1 strains like 648A and 584A were isolated which can break bivalent vaccination and cause acute transient paralysis as well as a significant increase in mortality within the first two weeks after infection^{15,37}. The appearance of vv+MDV has led to the introduction of the CVI988/Rispens vaccine in the U.S. which has been used in Europe since the 1970's^{21,22,36,40}.

The isolation of new highly virulent strains from vaccinated flocks has not only been reported in the U.S. but also in Europe. Changes of clinical signs and cellular tropism have been reported for strain C12/130³. The mechanisms underlying the steady increase in MDV virulence and the appearance of a more acute neuronal form of MD remain enigmatic, but vaccine formulations and application forms may have an impact on these phenomena³⁸. MDV exhibits high cell association in vitro and MD vaccines, except for some HVT formulations, represent living chicken cells infected with the agent which are kept in liquid nitrogen until application³⁰.

The in vitro cultivation of MDV is enormously tedious, laborious and difficult to standardize. It is now realized that some glycoproteins are essential for virus growth in cultured cells, that is, virus depleted of gE or gI for example does not spread in culture at all (Shumacher et al, J. Virol. 75: 11307-11318, 2001). An important cost factor in the production of vaccines for a prevention of MDV infection and MD in the domestic chicken is the use of cell cultures. To date the efficient growth of MDV is possible only on primary chicken or duck cells. Usually, the cell culture of choice for production of MDV vaccines is chicken embryo fibroblast cells (CEF)³⁸. These CEF are also used for isolation of MDV obtained from clinical cases in the form of peripheral blood cells (PBC) or after induction of lytic MDV replication from transformed tumor T cells. Alternatively, for isolation of virulent and not cell culture adapted virus strains or isolates, primary chicken kidney cells (CKC) or duck embryo fibroblasts (DEF) can be used. These cells proved superior to CEF for isolation of MDV from clinical samples. All known systems that support growth of MDV in culture rely on the production of primary cell cultures which have to be prepared continuously from embryonated eggs or even hatched birds in the case of CKC. The procedure of preparing primary cells not only is laborious but also causes high costs. This is reflected by the fact that approximately 30% of the cost of vaccine production is caused by the preparation of chicken embryo fibroblasts. Vaccine production is very dependent on a continuous and reliable supply of fertile eggs from specified pathogen free (SPF) flocks. SPF flocks are raised under special conditions and are regularly demonstrated to be free of avian pathogens. Any disruption in the supply of fertile SPF eggs would disrupt production of MDV vaccines.

Even though virulent and field MDV strains have been successfully propagated and isolated on chicken kidney (CKC) or duck embryo fibroblast cells, vaccine MDV or virulent strains, only efficiently grow on CEF cells after adaptation. Many attempts to propagate an MDV strain on continuous cell lines of avian or mammalian origin have failed. The failures were either caused by the loss of important properties of the virus after growth on heterologous cells or by the fact that abortive infections only could be demonstrated. In the last decade, however, the generation of cell lines constitutively infected with MDV have been reported. It was for example shown by Abujoub et al (Acta Virologica 43: 186-191, 1999 and in EP 0 775 743) that CHCC-OU2 cells, permanent derivatives of CEF cells, could be latently infected with virulent or avirulent MDV strains. However, long periods of MDV adaptation to the cell culture system used were necessary, in which changes in the genetic or antigenetic composition of the individual viruses could occur. With the CHCC-OU2 cell line, 4 weeks of culture were needed before MDV-specific plaques were visible. The resulting CEF cell lines OU2.1 and OU2.2 contained a latent form of virulent and tumor-inducing MDV as long as the cells were not confluent. The switch from latent to lytic infection was induced as soon as cell-to-cell contacts within the cultures were built. The cells are latently infected with only one strain and—upon confluency and by some mechanism no one really understands—lytic cycle replication is turned on. In conclusion, in this system one needs a special cell line for each virus one wants to propagate, one needs long times of adaptation and the cells are difficult to grow. Additionally the virulent Md11 strain grown on OU-OCL2 cells still was able to induce tumors in susceptible chicken lines. Recently, a continuous Vero cell line was tested for its susceptibility for MDV growth. It was reported that, after several rounds of blind passaging and 3 weeks of adaptation, very low titers of MDV serotype 1 (MDV-1) and serotype 3 (MDV-3) (i.e., low levels of infectious virus) could be produced. As outlined previously, long adaptation to cell culture systems are prone to result in unwanted genetic alterations which then can cause e.g., vaccine failures when vaccine strains have to be adapted to these cells. The poultry industry has always recognized the need for continuous cell lines that could be used for producing Marek's disease vaccines. Although many avian or mammalian cell lines have been developed (see, for example, EP 0 748 867 and EP 0 884 382), until the present invention, no continuous cell line developed could be an effective substitute for chicken embryo fibroblast (CEF) cells in vaccine production. The maximum titer of MDV recoverable from these continuous cell lines could not match the titer of virus recovered from primary cell lines. In addition many hypervirulent and virulent MDV strains could not be propagated on previously developed continuous cell lines nor on primary cell lines.

### DISCLOSURE OF THE INVENTION

Provided is a method for producing a continuous cell line, essentially free of an mammalian or avian virus, capable of supporting the growth of a herpes virus strain comprising infecting or transfecting a cell with a vector comprising a nucleic acid fragment derived of a herpes virus, preferably an alpha herpes virus, and culturing the infected or transfected cell or progeny thereof under conditions suitable for the expression of the nucleic acid and propagation of the cell or progeny thereof. Considering that the cell line is essentially free of virus it is a prerequisite that such a nucleic acid does not encode for a replicating herpes virus itself, to allow for later infection with a herpes virus strain to be propagated. Therefore, the continuous cell line as provided here is not in itself a (latent) virus producer, but is used to cultivate or grow a herpes virus after inoculation therewith to sufficient titres to for example produce a vaccine. It is for example sufficient that the nucleic acid or fragment thereof of a herpes virus comprises a nucleic acid encoding a structural protein or glycoprotein or part thereof. Examples of herpes viruses include Varicella Zoster virus, cytomegalovirus, Epstein-Barr virus, herpes simplex virus, etc. Thus, the present invention, among other things, provides a continuous cell line that can support the growth of herpes viruses, including hypervirulent, virulent and avirulent MDV strains without need for adaptation of the strains for vaccine production.

### BRIEF DESCRIPTION OF THE FIGURES

FIG. 1: Schematic representation of the MDV-1 genome and location of the gE open reading frame in the unique short region (Us). Shown is the organization of the approximately 180 kbp MDV-1 genome and the BamHI restriction map. The gE open reading frame was amplified by PCR and cloned into pcDNA3 vector (Invitrogen) under the control of the human cytomegalovirus immediate early promoter/enhancer. Scales are given in kbp or bp.

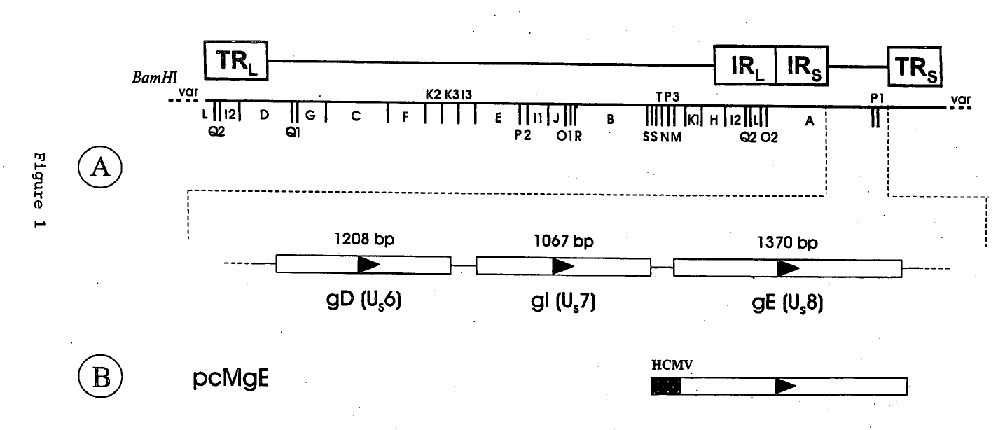

FIG. 2: Detection of gE expression by SOgE cells by IIIF using a rabbit polyclonal gE specific antiserum³⁰. Whereas reactivity of SOgE cells with the antibody was readily detected, no reactivity of the gE-specific serum with QM7 cells was observed. The individual views are 1,000×650 μm in size.

FIG. 3: Polymerase chain reaction of SOgE cells. SOgE cells were left uninfected or were infected with MDV-1 strains 584Ap80C, CVI988 or RB1B. Whereas gE-specific sequences were detected in all infected and uninfected SOgE cells, a gB-specific amplification product was obtained in infected cells only. In QM7 cells neither gB nor gE was detectable. The sizes of the amplification products are given. Specificity of the amplified products was verified using Digoxigenin-labeled gB or gE sequences as probes. Detection of DNA-DNA hybrids was done using CSPD™ (Roche Biochemicals) and enhanced chemoluminescence according to the manufacturer's instructions.

FIG. 4: Growth of gE-negative and BAC20 virus on gE-expressing SOgE cells or QM7 cells. After transfection of 20ΔgE or BAC20 DNA (Schumacher et al., 2000,2001) into the respective cells, 20ΔgE plaque formation was visible from day 3 after transfection in SOgE but not in QM7 cells. In QM7 cells, only single infected cells but no plaque formation was observed in case of 20ΔgE virus. BAC20 virus caused very small plaques in QM7 cells but large plaques on recombinant SOgE cells. The individual views are 1,000×650 μm in size (lower panels) or 300×200 μm in size (upper panels).

FIG. 5: Plaques of MDV-1 strains 584Ap80C, RB1B and CVI988 on CEF and SOgE cells. Plaque formation induced by the vaccine strains 584Ap80C, CVI988 and the virulent MDV-1 strain RB1B were readily observed after plating on SOgE cells. Virus plaque formation on CEF cells was also observed in case of 584Ap80C, CVI988 and RB1B. The plaques shown were fixed at Day 4 after infection of 1×10⁶ cells with 100 PFU of the indicated viruses. The individual views are 1,500×1,000 μm in size.

FIG. 6: ELISA titers of chickens immunized with CVI988 produced on recombinant SOgE cells or on CEF. Day—12 is the Day of immunization, on Day 0 animals were challenged with hypervirulent MDV-1 strain EU1. Titers of MDV-1-specific antibodies in plasma. All birds of each group were bled on the indicated days and plasma samples of two birds were pooled. Plasma was diluted in log₂ steps starting at a 1:100 dilution. Titers are expressed as the dilution in which the A₄₅₀ₙₘ. after reaction with MDV-infected cell lysates exceeded that with uninfected cell lysates by 3 standard deviations. Symbols for individual groups are explained.

### DETAILED DESCRIPTION OF THE INVENTION

In a preferred embodiment, the invention provides the use of a cell infected or transfected with a vector comprising a nucleic acid or fragment thereof of a glycoprotein E (gE) gene or functional fragment thereof. In a preferred embodiment, the invention provides a nucleic acid wherein the nucleic acid comprises an MDV glycoprotein gE gene or functional fragment thereof or a glycoprotein gE gene equivalent. Preferably, the glycoprotein gE gene is derived from an alpha herpes virus. As used herein, “a functional fragment thereof” refers to a gene/nucleic acid fragment whose expressed product retains the property of supporting/sustaining virus growth, preferably a herpes virus growth in the continuous cell line. A glycoprotein gE gene “equivalent”, as used herein, is any like gene (i.e., functional equivalent) for example a glycoprotein gE gene obtained from another herpes virus, such as an alpha herpes virus whose expressed product has the property of supporting/sustaining virus growth in the continuous cell line.

In another preferred embodiment, the invention provides a nucleic acid wherein the nucleic acid comprises an MDV glycoprotein gL gene or functional fragment thereof or a glycoprotein gL gene equivalent. Preferably, the glycoprotein gL gene is derived from an alpha-herpes virus. As used herein, “a functional fragment thereof” refers to a gene/nucleic acid fragment whose expressed product retains the property of supporting/sustaining virus growth, preferably a herpes virus growth in the continuous cell line. A glycoprotein gL gene “equivalent” as used herein is any like gene (i.e., functional equivalent) for example a glycoprotein gL gene obtained from another herpes virus, such as an alpha herpes virus whose expressed product has the property of supporting/sustaining virus growth in the continuous cell line.

In another preferred embodiment, the invention provides a nucleic acid wherein the nucleic acid comprises an MDV glycoprotein gI gene or functional fragment thereof or a glycoprotein gI gene equivalent. Preferably, the glycoprotein gI gene is derived from an alpha herpes virus. As used herein “a functional fragment thereof” refers to a gene/nucleic acid fragment whose expressed product retains the property of supporting/sustaining virus growth, preferably a herpes virus growth in the continuous cell line. A glycoprotein gI gene “equivalent” as used herein is any like gene (i.e., functional equivalent) for example a glycoprotein gI gene obtained from another herpes virus, such as an alpha herpes virus whose expressed product has the property of supporting/sustaining virus growth in the continuous cell line. In a preferred embodiment, the invention provides a method for producing a continuous cell line capable of supporting the growth of an MDV strain comprising infecting or transfecting a cell with a vector comprising a nucleic acid or fragment thereof of an MDV strain and culturing the infected or transfected cell or progeny thereof under conditions suitable for the expression of the nucleic acid and propagation of the cell or progeny thereof. It is a requisite that such a nucleic acid does not encode for replicating MDV itself, to allow for later infection with a herpes virus strain to be propagated.

A “continuous cell line” as used herein is an established/sustainable cell line, which can be grown without restrictions and can undergo at least 10 passages. Again, it is desired that the continuous cell line, albeit provided with a nucleic acid fragment derived from a herpes virus, be free of mammalian and avian viruses, as, incidentally, was found to be not the case for the quail cell line QT-35 which turned out to be latently infected with an MDV. By use of the term “cell or progeny thereof” it is understood that the cell can be cultured and expanded from a single cell to produce a cell line using several commercially available culture media, under known conditions suitable for propagating avian cells. By “infection” or “transfection” is meant the DNA transfer of virus or fragment(s) thereof into avian or mammalian cells. Methods for gene transfer into cells are known to those skilled in the art. For example, the method of transfection may comprise known techniques used to transfect DNA into cells, such as calcium phosphate precipitation, polybrene, DEAE-dextran, LIPOFECTIN, electroporation, or protoplast fusion.

A “vector” as used herein comprises any gene delivery vehicle which is capable of delivering nucleic acid to the cell, such as for example a plasmid vector (pcDNA3, Invitrogen). Nucleic acid as used herein refers to a nucleic acid sequence of a herpes virus, preferably an alpha herpes virus, and even more preferred an MDV such as for example a gene equivalent to the glycoprotein E (gE) of alpha herpes viruses or a fragment thereof. The preferred MDV strain is selected from an MDV oncogenic serotype-1 (MDV-1) and/or nononcogenic serotype-2 (MDV-2) and/or nononcogenic serotype-3, turkey herpes virus (HVT). Such a nucleic acid according to the invention may be expressed constitutively or transiently under the control of the cell's own regulatory elements or heterologous regulatory elements (i.e., constitutively active regulatory elements or inducible regulatory elements). In a preferred embodiment, the nucleic acid according to the invention, its expressed product has the functional property of supporting/sustaining the growth of a virus, preferably avian viruses, (e.g., MDV, Varicella Zoster virus, Newcaster Disease Virus (NDV), Infectious Bursal Disease Virus (IBDV), Infectious Bronchitis Virus (IBV), Chicken Anemia Virus (CAV), Infectious Laryngotracheitis Virus (ILV), Avian Leukosis Virus (ALV), Reticuloendotheliosis Virus (REV) and Avian Influenza Virus (AIV), etc.) in the continuous cell line.

Various culturing conditions for the cell line, including media formulations with regard to specific nutrients, oxygen, tension, carbon dioxide and reduced serum levels, can be selected and optimized by one of skill in the art. Preferably, the infected or transfected cell according to the invention comprises a vertebrate cell. Even more preferred the infected or transfected cell comprises an avian cell. For example the cell may be derived from muscle tissue, e.g., a muscle myoblast cell. Any muscle myoblast cell may be utilized in accordance with the present invention for example the muscle myoblast cell may be obtained from human, primate, chicken, quail, turkey, goose, pigeon, pheasant, bobwhite muscle tissue. Muscle cell lines can be cultured and maintained using known vertebrate cell culture techniques. In a preferred embodiment, the myoblast cell is obtained from quail such as for example a QM cell. A preferred QM cell is a QM7 cell deposited as ATCC CRL-1632.

In a preferred embodiment, the invention provides a nucleic acid wherein the nucleic acid comprises an MDV glycoprotein gE gene or functional fragment thereof or a glycoprotein gE gene equivalent. Preferably, the glycoprotein gE gene is derived from an MDV serotype 1 and/or serotype 2 and/or serotype 3. As used herein “a functional fragment thereof” refers to a gene/nucleic acid fragment whose expressed product retains the property of supporting/sustaining virus growth, preferably an MDV grown in the continuous cell line. A glycoprotein gE gene “equivalent” as used herein is any like gene (i.e., functional equivalent) for example a glycoprotein gE gene obtained from another herpes virus, such as an alpha herpes virus whose expressed product has the property of supporting/sustaining or enhancing virus growth in the continuous cell line. In another preferred embodiment, the MDV comprises an avirulent MDV strain such as for example Rispens CVI988. The invention encompasses all attenuated MDV/vaccine strains that are capable of replication in the continuous cell line. The invention provides a method for producing a continuous cell line capable of supporting the growth of an MDV strain comprising infecting or transfecting a cell with a vector comprising a nucleic acid or fragment thereof of an MDV strain and culturing the infected or transfected cell or progeny thereof under conditions suitable for the expression of the nucleic acid and propagation of the cell or progeny thereof, wherein the continuous cell line is capable of supporting the growth of an MDV strain without adaptation of the virus. “Without adaption” as used herein means that the continuous (i.e., permanent/sustainable) cell line according to the invention is capable of supporting the growth and productive infection of an MDV at high titers (i.e., at a level comparable or even superior to that of a primary cell line, for example, chicken embryo fibroblast cells) after 1 or 2 passages.

In yet another embodiment, the invention provides a cell or progeny thereof obtainable by a method according to the invention. Furthermore, the invention provides a cell preparation obtained from a cell or progeny thereof according to the invention. Preferably, the cell or progeny thereof or cell lysate is infected or transfected with an MDV serotype 1 attenuated strain. The invention provides use of a cell infected or transfected with a vector comprising a nucleic acid or fragment thereof of an MDV strain according to the invention and/or a preparation derived therefrom for the preparation of a vaccine capable of inducing some measure of protection against a disease (i.e., to produce immunity) in a vertebrate, preferably an avian species. The present invention relates also to a method for the preparation of MDV in quantities suitable for vaccine purposes. The invention provides a method to prepare a vaccine capable of inducing an immune response against disease in a vertebrate, preferably an avian species, comprising culturing a cell according to the invention and harvesting cell culture components therefrom. It is also understood that the MDV strain can be maintained as a non-lytic or a lytic infection in the cell line (i.e., SOgE), and the cell line according to the invention is able to infect vertebrates, preferably avians in vivo, in order to produce immune protection against Marek's Disease

Furthermore the invention provides a method for producing both monovalent and multivalent vaccines for protecting vertebrates, preferably avian species against infection by disease causing agents, i.e., in addition to MDV. It is understood that the cell line according to the invention (i.e., SOgE) can be infected or transfected with a vector such as an MDV vector comprising an MDV genome or parts thereof including genes encoding one or more heterologous proteins or polypeptides (i.e., antigenic peptides of disease causing agents) which are useful against vertebrate disease causing agents other than an MDV. Examples of disease causing agents which may be useful for producing vaccines against include Newcastle Disease Virus (NDV), Infectious Bursal Disease Virus (IBDV), Infectious Bronchitis Virus (IBV), Chicken Anemia Virus (CAV), Infectious Laryngotracheitis Virus (ILV), Avian Leukosis Virus (ALV), Turkey Astrovirus Reticuloendotheliosis Virus (RV) and Avian Influenza Virus (AIV). It is understood that virtually any heterologous gene sequence can be constructed in the vector comprising the nucleic acid according to the invention for use in the preparation of vaccines, preferably multivalent vaccines. It is preferable that such a heterologous gene sequence will encompass a gene product that can serve to boost or activate the host's cellular and/or humoral immune response, or a gene product that encodes an epitope that induces a protective immune response to any variety of pathogens (preferably vertebrate pathogens, even more preferred avian pathogens), or antigens that bind neutralizing antibodies. For example genes or gene fragments encoding antigenic peptides of one or more of the three serotypes of MDV. Such antigenic peptides may be used in a multivalent vaccine to be administered to a vertebrate so as to confer a complete protective effect against infection with an MDV.

The invention further provides a vaccine obtainable by a method according to the invention. Such vaccines can be prepared by methods well known to those skilled in the art of preparation of vaccines. Vaccines may take the form of suspensions of a cell line infected or transfected with the vector comprising a nucleic acid or fragment thereof of an MDV strain according to the invention, infected with and supporting the productive replication of a vaccine strain/attenuated strain of an MDV, preferably serotype 1 and/or serotype 2 and/or serotype 3 (i.e., singly or monovalent vaccines) or in combinations thereof (i.e., multivalent vaccines)), and/or other viruses, like herpes viruses. Additionally, a vaccine according to the invention may be made from cell free virus suspensions made from sonicated cells, for example, SogE cells, and/or cell lysates or components of lysates infected with and supporting the productive infection of a vaccine strain/attenuated strain of an MDV preferably serotype 1 and/or serotype 2 and/or serotype 3 (singly or in combination thereof) and/or other viruses, like herpes viruses. It is understood that a vector according to the invention may be engineered using recombinant techniques within the skill of those in the art to encode heterologous proteins which are known to boost a vertebrate immune response (e.g., capable of stimulating class 1 or 2 major histocompatibility complex molecule presentation), thus enhancing the overall protective effect of an administered vaccine.

The invention provides the use of a cell infected or transfected with a vector comprising a nucleic acid or fragment thereof of an MDV strain according to the invention for the generation and/or maintenance and/or isolation of a herpes virus, preferably an alpha herpes virus. Examples of herpes viruses include Varicella Zoster virus, cytomegalovirus, Epstein-Barr virus, herpes simplex virus etc. In a preferred embodiment, the invention provides the use of a cell infected or transfected with a vector comprising a nucleic acid or fragment thereof of an MDV strain according to the invention for the generation and/or maintenance and/or isolation of a hypervirulent and/or virulent and/or avirulent MDV strain. Such a cell can be used as a suitable substrate for the propagation of hypervirulent strains, such as the EU1, 648A and 584A strain, and/or virulent strains such as the RB1B strain and/or avirulent strains such as 584Ap80C and CVI988. The invention encompasses all MDV strains selected from the group consisting of serotype 1, serotype 2, serotype 3. Serotype 1 includes all pathogenic strains and their attenuated derivatives. Serotype 2 consists of naturally avirulent chicken viruses, while serotype 3, also known as Herpes virus of Turkeys (HVT), includes avirulent turkey viruses that are capable of replication in chickens. It is understood that the MDV strain may comprise a natural or a genetically modified strain.

The present invention encompasses a method to generate and/or isolate and/or maintain a very virulent (vv), a very virulent plus (vv+), a hypervirulent and/or virulent and/or avirulent MDV strain comprising infecting a cell capable of supporting the growth of an MDV strain according to the invention with a herpes virus and culturing the cell under conditions suitable for the propagation of the cell and the generation, maintenance and isolation of the herpes virus. The invention furthermore provides a herpes virus obtainable by a method according to the invention.

In a preferred embodiment, the invention provides a method to generate and/or isolate and/or maintain very virulent (vv), a very virulent plus (vv+), a hypervirulent and/or virulent and/or avirulent MDV strain comprising infecting a cell capable of supporting the growth of an MDV strain according to the invention with a very virulent (vv), a very virulent plus (vv+), hypervirulent and/or virulent and/or avirulent MDV strain and culturing the cell under conditions suitable for the propagation of the cell and the generation, maintenance and isolation of the very virulent (vv), a very virulent plus (vv+), hypervirulent and/or virulent and/or avirulent MDV strain. Suitable conditions for culturing vertebrate/avian cells are known in the art. The invention furthermore provides a very virulent (vv), a very virulent plus (vv+), hypervirulent and/or virulent and/or avirulent MDV strain obtainable by a method according to the invention.

Additionally, it is preferred that the cell capable of supporting the growth of an MDV strain according to the invention is cultured in the presence of geneticin (G-418), derivative or analog thereof or any other drug that is nontoxic in the presence of a neomycin resistance gene and can be used for selection. A derivative of G-418 as used herein is a substance derived from G-418 with like properties. An analog of G-418 as used herein is a substance possessing a chemical structure and chemical properties similar to that of G-418 (Gibco, Life Technologies).

The invention further provides the use of a cell capable of supporting the growth of an MDV strain according to the invention for the production of a diagnostic antigen of a herpes virus, preferably an alpha herpes virus and even more preferred an MDV. It is understood that virtually any heterologous gene sequence can be constructed in the vector comprising the nucleic acid according to the invention for use in the preparation of a diagnostic antigen. For example genes or gene fragments encoding non common antigenic peptides of one or more of the three serotypes of MDV, or gene or gene fragments encoding specific antigenic peptides of other herpes viruses. Such specific viral antigens may be used to diagnose a specific viral infection in a bird and are also useful for vaccine production. Furthermore the invention provides a method for the production of a diagnostic antigen of an MDV comprising providing a cell according to the invention infected with and capable of supporting the growth of an MDV strain according to the invention and isolating components therefrom.

The invention further provides a vaccine according to the invention for the preparation of a medicament for the prevention and/or treatment of a vertebrate disease. Preferably, the vertebrate disease is an avian disease, even more preferred an MDV associated disease. Suitable basis for a medicament are known in the art. The invention includes both prophylactic and therapeutic vaccines. The invention further provides a method for protecting a vertebrate against a disease comprising administering to the vertebrate a vaccine according to the invention with a pharmaceutically acceptable carrier to a suitable recipient. The invention further provides a method for immunizing a vertebrate (e.g., animal, human, poultry) against infectious herpes virus comprising administering to a vertebrate an effective immunizing dose of a vaccine according to the present invention. Either a live recombinant viral vaccine or an inactivated recombinant viral vaccine can be formulated. Production of live virus vaccine formulations can be accomplished using conventional methods involving propagation of the virus in a cell line according to the invention followed by administration as a medicament to a vertebrate. Vaccines may be administered to a vertebrate by any of the methods well known to those skilled in the art, for example by intramuscular, subcutaneous, intraabdominal, intravenous, or in-ovo injection. It is preferable to introduce the virus vaccine formulation via the natural route of infection of the pathogen for which the vaccine is designed. Alternatively such a vaccine may be administered oculo-nasally or orally. In order to prepare inactivated vaccines, the virus may be grown in cell line according to the invention, inactivated by for example formaldehyde or beta-propiolactone. Inactivated viruses may be formulated with a suitable adjuvant in order to enhance the immunological response. Such adjuvants may include, but are not limited to, mineral gels, e.g., aluminum hydroxide; surface active substances such as lysolecithin, pluronic polyols, polyanions; peptides; oil emulsions; and potentially useful adjuvants such as BCG and Corynebacterium parvum. Pharmaceutically acceptable carriers are well known in the art and include, but are not limited to, saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, dimethylsulfoxide etc. The carrier is more preferably sterile. A suitable recipient is a vertebrate, preferably an avian species.

The invention is further described with the aid of the following illustrative examples.

### EXAMPLE 1

Animals. White Lohmann selected leghorns (LSL) (Lohmann, Cuxhaven, Germany) were used and wing-banded on the day of hatch. Birds were kept in cages, and received food and water ad libitum.

Cells and viruses. Chicken embryo fibroblast cells (CEF) were prepared as described previously (Schumacher et al., 2000). Quail muscle QM7 cells (ATCC cell number CRL-1632) were used for preparation of the permanent cell line. The MDV strains used were CVI988 (MarekVac forte®, Lohmann, Cuxhaven, Germany), 584Ap80C³⁷, RB1B (kindly provided by Dr. T. F. Davison, Compton, UK), and EU1. Strain 584Ap80C or its US2-negative derivative BAC20 virus reconstituted from an infectious BAC clone of MDV (Schumacher et al., 2000) were propagated on CEF²⁹. MDV strain EU1 is a virus isolated in 1992 from a flock in Italy which had been immunized with an HVT vaccine. EU1 was shown to be free of chicken infectious anemia virus, avian leukosis and reticuloendotheliosis viruses, and infectious bursal disease virus by PCR, and could not be propagated on CEF. For preparation of virus, strain EU1 was injected intramuscularly (i.m.) into 10 chickens. At day 10 after infection, peripheral blood (PBMC) and spleen mononuclear cells were harvested from infected birds by Histopaque® density centrifugation (Sigma, Munich, Germany) and stored in liquid nitrogen.

Plasmid. The glycoprotein E open reading frame, the US8 homologous gene 17, 33 of MDV vaccine strain Rispens CVI988 was amplified by polymerase chain reaction (PCR) using 100 pmol each of the 5′-primer 5′-CATAAGCATGCGAGTCAGCGTCATAATGTG-3′. (SEQ ID NO:______) and the 3′-primer 5′-CAAGGGCCCATCAGTGGTATAAATCTAAGC-3′ (SEQ ID NO:______). The resulting 1.4 kbp fragment was cleaved with restriction endonucleases BamHI and EcoRI and cloned in the same sites of vector pcDNA3 (Invitrogen) leading to recombinant vector pcMgE³⁰.

PCR analysis. One PCR assay targeted the gE open reading frame (see above), the other PCR assay performed on infected cells targeted the gB gene of MDV (Lee et al., 2000). The PCR assay²⁵ targeting the MDV gB gene^{17,33} was done with 100 pmol each of forward (5′-GCATATCAGCCTGTTCTATC-3′) (SEQ ID NO:______) and reverse primer (5′-AACCAATGGTCGGCTATAAC-3′) (SEQ ID NO:______). In both protocols, the respective primers were mixed with DNA, and 35 cycles (95° C. 30 s, 50° C. 30 s, 72° C. 30 s) were run. The specificity of PCR products was confirmed by Southern blotting using Digoxigenin-labeled gB or gE sequences as a probe²⁹.

Indirect immunofluorescence analysis. For indirect immunofluorescence analyses (IIF), cells were grown on 6-well plates (Greiner) or on glass coverslips. Cells were fixed with 90% acetone at various times after infection, IIF was done exactly as described, and samples were analyzed by conventional fluorescence microscopy (29,30). The antibodies used were anti-gB monoclonal antibody (mab) 2K11; kindly provided by Dr. J.-F. Vautherot, INRA, Nouzilly, France) or a convalescent serum from a chicken infected with MDV-1 (anti-MDVI)²⁹.

ELISA Procedure to Determine MDV-1 Antibodies

MDV-1-specific antibodies in plasma were determined by enzyme-linked immunosorbant assays (ELISA). The antigen consisted of a lysate of 5×10⁷ BAC20-infected CEF harvested 5 days after infection by freeze-thawing. The lysate (5 ml in PBS) was sonicated for 60 s at 60 W, and cell debris was removed by centrifugation (10,000 g, 10 min, 4° C.). A lysate of 5×10⁷ uninfected CEF was used as the negative control. ELISA plates (Nunc) were coated with 100 μl of lysates (final concentration: 5 μl lysate/ml) for 16 hours at 4° C. Plasma samples were diluted in log₂-steps starting with a 1:100 dilution and added to plates for 60 min at 25° C. after blocking using 2% skim milk. After thorough washing with PBS, 100 μl anti-chicken IgG peroxidase conjugate (Sigma) was added for 30 min at 25° C. After final washing with PBS, 100 μl TMB substrate solution (Sigma) was added for 10 min before the reaction was stopped with 2 M H₂SO₄. Absorbances at 450 nm (A₄₅₀) were read (TecanSPECTRA, Crailsheim, Germany), and end-point antibody titers were determined as previously²⁰.

### EXAMPLE 2

### Generation of a Cell Line Constitutively Expressing Glycoprotein E of MDV Vaccine Strain CVI988

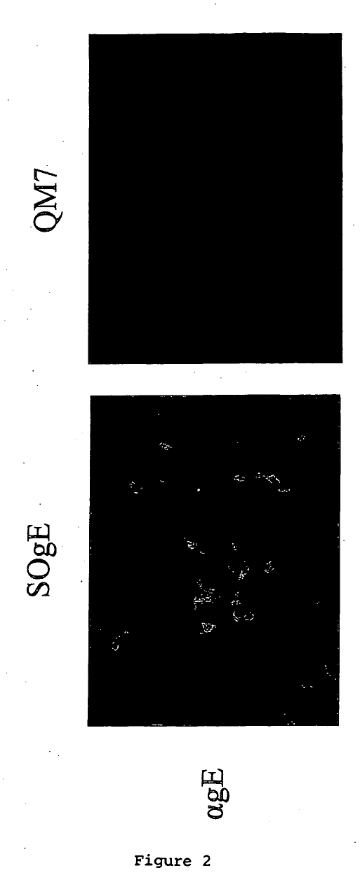

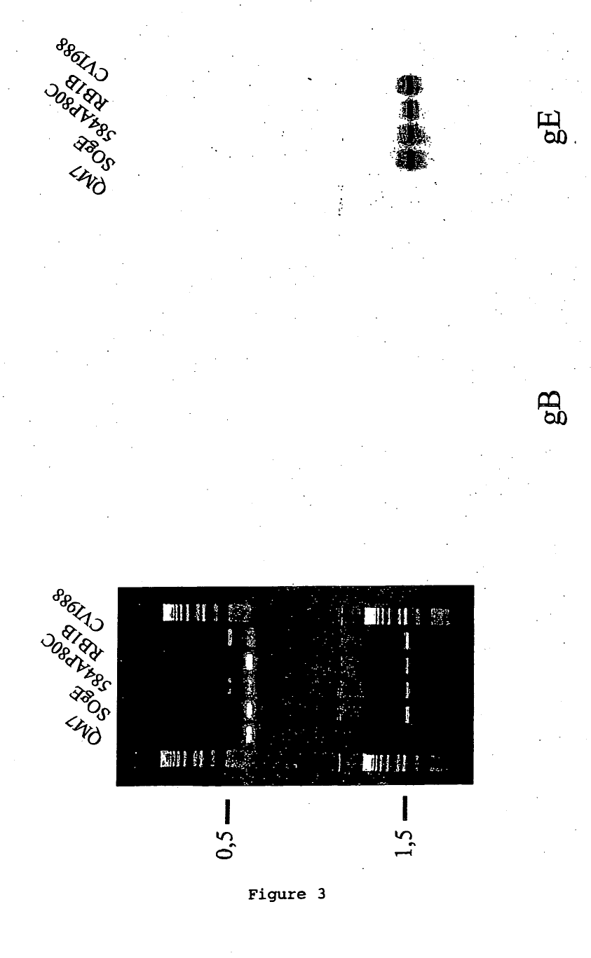

QM7 cells were transfected by the calcium phosphate precipitation method as described earlier²⁹. QM7 cells were grown on 6-well plates until approximately 70% confluency and transfected with 10 μg of recombinant plasmid pcMgE³⁰. Transfected cells were overlaid with DMEM medium containing 5% of FCS and 1000 μg per ml of G-418 (Gibco-BRL). After 2 weeks of cell culture in the presence of the antibiotic, single cell clones were picked into 96-well plates. After cell clones had grown to confluency in 96-well plates, they were split 1:2 and IIF using the convalescent MDVI serum was performed on the cell clones. A cell clone in which virtually every cell expressed MDV gE was identified (FIG. 2), and termed SOgE. SOgE cells were expanded and checked for gE expression in weekly intervals. In the presence of G-418, gE expression proved stable for more than 15 weeks. In the absence of the drug, gE expression in SOgE cells was detected for up to 10 weeks. Presence of MDV gE DNA in SOgE but not in QM7 cells was confirmed by PCR analysis of DNA prepared from 1×10⁷ cells each (FIG. 3).

### EXAMPLE 3

### SOgE Cells Express Functional MDV gE

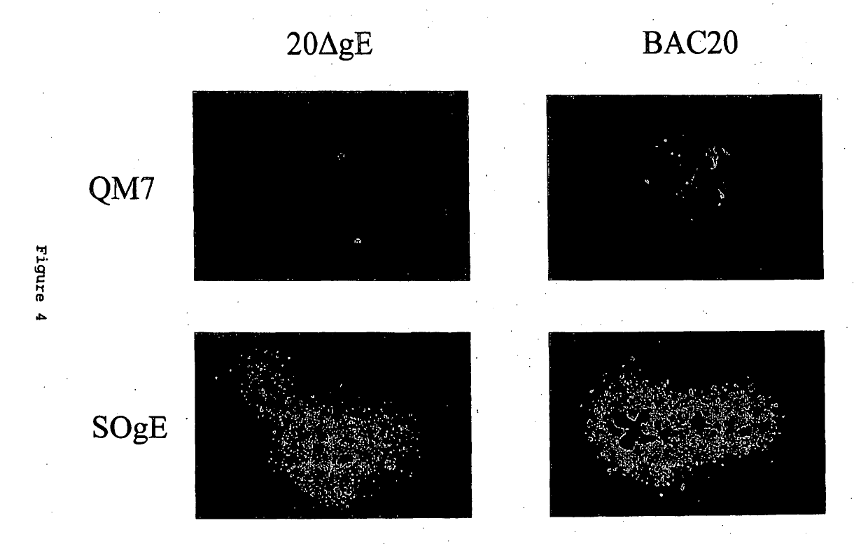

The question of expression of functional MDV gE by the generated cell line was addressed by the analysis of growth of a gE-negative MDV on SOgE cells. Glycoprotein E has been shown to be essential for MDV growth in vitro³⁰. Therefore, 20ΔgE DNA encoding gE-negative BAC20 virus (Schumacher et al., 2000) was transfected into SOgE or QM7 cells and plaque formation was monitored. Whereas 20ΔgE plaques were readily observed on SOgE cells after IIF using anti-gB mab 2K11, only single infected cells could be visualized with the mab on parental QM7 cells (FIG. 4). These results confirmed that functional gE was produced by SOgE cells which was able to trans-complement the essential MDV-1 gE in a gE-deficient virus.

### EXAMPLE 4

### SOgE Cells Support Growth of Several MDV-1 Strains

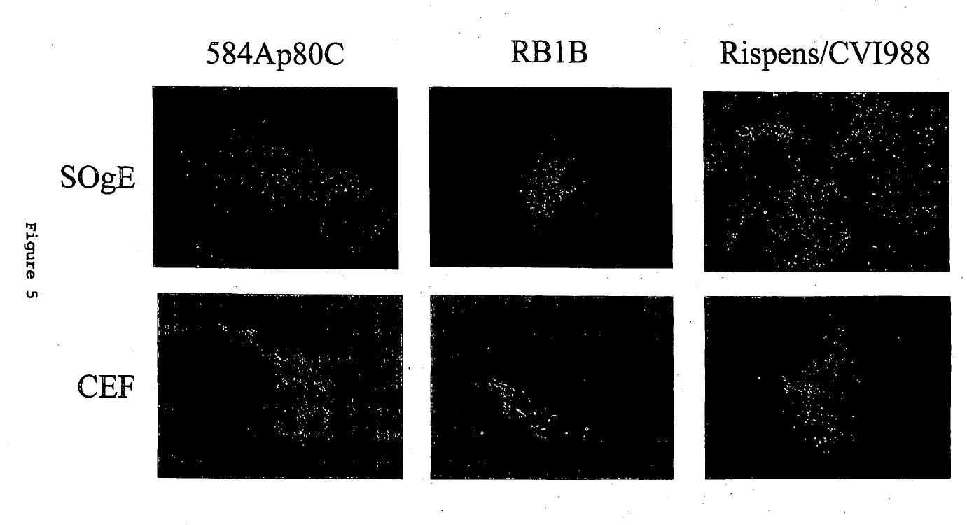

The size of 20ΔgE plaques on SOgE cells appeared much larger than that of BAC20 virus on QM7 cells or that of a gB-negative virus mutant on gB expressing QM7 cells²⁹. This unexpected observation was addressed in further experiments. Plaque sizes of the avirulent MDV-1 strains 584Ap80C and CVI988 as well as the virulent RB1B and hypervirulent strain were assessed on CEF, QM7 and SOgE cells. This was done by co-seeding of infected CEF or PBMC (EU1) with the respective cells. It could be demonstrated that co-seeding of SOgE cells with CEF cells infected with 584Ap80C, CVI988, or RB1B at low multiplicity of infection (MOI=0.0001; i.e., 100 PFU per 1×10⁶ cells) led to plaque formation (FIG. 5). The plaque sizes were comparable to those on CEF cells (FIG. 5). In addition, it was possible to directly reconstitute infectious MDV-1 from viral DNA or Escherichia coli-derived cloned viral DNA (BAC20),²⁹ after transfection of SOgE cells (FIG. 5). Thus, direct adaptation of MDV to SOgE cells without lengthy cell culture passaging is possible, which is a major advantage for both vaccine production and isolation or generation of virulent and hypervirulent MDV-1 for animal experiments.

The above described experiments had suggested that SOgE cells represent a permanent cell substrate for efficient propagation of both avirulent (vaccine) MDV strains and virulent, very virulent and hypervirulent MDV-1 strains. Because especially production of MD vaccines could be facilitated using a permanent cell line allowing propagation of MDV vaccine strains, CVI988 DNA was transfected into CEF, QM7, or SOgE cells. At 6 days after transfection when virus plaques were visible, cells were harvested and 1×10³ infected cells were co-seeded with 1×10⁷ uninfected cells of the matching cell type. At 5 days after infection of this large number of cells, infected cells were trypsinized and titrated in tenfold dilutions on freshly prepared CEF, QM7 or SOgE cells. At 4 days after titration, cells were fixed and numbers of virus plaques were determined after IIF staining using the 2K11 anti-gB mab antibody. It could be shown that mean CVI988 titers on SOgE cells reached 1.8×10⁶ PFU whereas titers of only 3.2×10³ were obtained on QM7 cells. The titers on SOgE cells were virtually identical to those on primary CEF cells. From the results of the plaque size determinations and the titration experiments we concluded that propagation of virulent and avirulent vaccine MDV strains on SOgE cells, which constitutively express MDV strain CVI988 gE, was as effective as or even superior to propagation on primary CEF cells.

### EXAMPLE 5

### SOgE Cells Do Not Cause Tumors in 1-Day Old Chickens

The fact that SOgE cells were derived from a chemically induced quail tumor raised the remote possibility that it may cause tumors in chickens after systemic application of whole cell preparations. To address this important issue, a total of 18 chickens was inoculated with either SOgE cells (12 chickens) or parental QM7 cells (6 chickens). Each individual chicken received 1×10⁶ cells by the intramuscular and 1×10⁶ cells by the intraabdominal route (Table 1). The fate of the inoculated chickens was followed for a total of 12 weeks, when post-mortem examination was performed. None of the chickens exhibited any clinical signs during the course of the experiment. Additionally, all chickens appeared in a good nutrition state and without any sign of tumor formation at the post-mortem examination. From these results we concluded that recombinant MDV gE-expressing SOgE cells as well as parental QM7 cells do not cause tumors in chickens, even when approximately 100- to 1000-fold more cells compared to a vaccine dose are administered. Therefore, we considered SOgE cells as safe for the production of MDV or combined vaccines.

### EXAMPLE 6

### EXAMPLE 6

### Protection of Chickens Against Marek's Disease Using SOgE Produced Vaccine

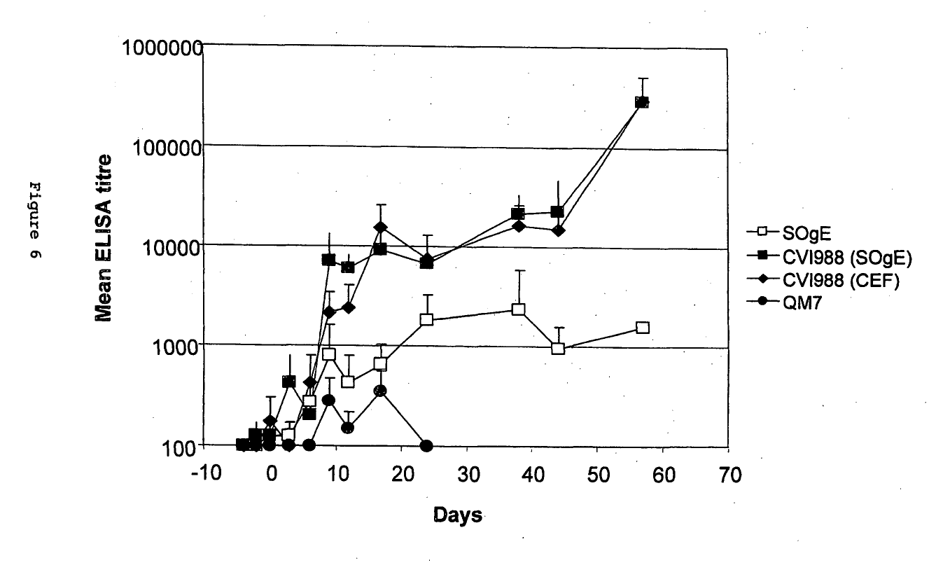

To compare the protective capacity of the vaccine strain Rispens CVI988 propagated on SOgE cells with that produced conventionally on CEF cells, animal experiments were performed. One-day-old chickens received 1×10⁶ SOgE cells (Group 1, Table 2), 1×10⁶ parental QM7 cells (Group 4, Table 1) or 1×10³ plaque-forming units of CVI988 produced on SOgE (Group 2, Table 2) or CEF cells (Group 3, Table 2). To avoid cross-contamination, vaccine virus was produced after transfection of CVI988 DNA isolated from CEF into the SOgE or CEF cells. Transfected cells were co-seeded with fresh uninfected cells and vaccine virus was harvested on Day 5 p.i. when complete cytopathic effect had developed. Immunized birds were challenge-infected with hypervirulent MDV-1 strain EU1 on Day 12 after immunization. In the mock-immunized animals (QM7 cells, group 4), chickens suffered from MD as early as 7 days after infection and two birds died on Day 9 and 10, respectively. By Day 37 p.i. all birds had died as a consequence of the infection (Table 2). In the SOgE-immunized animals, 4 chickens died as a consequence of the infection, but 3 birds survived until termination of the experiment on Day 70 after infection (Table 2). However, the 3 surviving birds exhibited MD as evidenced by gross pathology examination and the detection of tumours of inner organs (Table 2). In stark contrast, chickens immunized with CVI988, which was produced either on SOgE or on CEF cells, did not die from MD until the termination of the experiment (Table 2). In one bird each of Group 2 and 3, however, signs of MD were identified by gross pathology (Table 2). Serological anti-MDV-1 responses in immunized and challenged birds were followed by performing ELISA testings. It could be demonstrated that protected birds exhibited ELISA antibody that were gradually rising with time after challenge infection, whereas chickens inoculated with SOgE or QM7 cells did not develop high antibody titers. Rather, antibody titers fell at later times after infection or remained virtually constant (FIG. 6). From the results it was concluded that the MD CVI988 vaccine which was produced on permanent SOgE cells provided as good a protection as that produced conventionally on CEF cells. In addition, protection against MD was coincident with gradually rising anti-MDV-1 antibodies, which may indicate that a permanent boost of the chicken immune systems by either lytically replicating vaccine virus or low level replication of EU1, which may be controlled by the presence of vaccine virus, is a prerequisite for protection against tumourigenic MD.

### References

1. Adler, H., M. Messerle, M. Wagner, and U. H. Koszinowski. 2000. Cloning and mutagenesis of the murine gammaherpes virus 68 genome as an infectious bacterial artificial chromosome. J. Virol. 74:6964-6974.

2. Adler, H. E. and A. J. DaMassa. 1979. Toxicity of endotoxin to chicks. Avian Dis. 23:174-178.

3. Barrow, A. and K. Venugopal. 1999. Molecular characteristics of very virulent European MDV isolates. Acta Virol. 43:90-93.

4. Benton, W. J. and M. S. Cover. 1957. The increased incidence of visceral lymphomatosis in broiler and replacement birds. Avian Dis. 1:320-327.

5. Calnek, B. W. 2001. Pathogenesis of Marek's disease virus infection. Curr. Top. Microbiol. Immunol. 255:25-55.

6. Calnek, B. W., K. A. Schat, M. C. Peckham, and J. Fabricant. 1983. Field trials with a bivalent vaccine (HVT and SB-1) against Marek's disease. Avian Dis. 27:844-849.

7. Cui, Z., A. Qin, L. F. Lee, P. Wu, and H. J. Kung. 1999. Construction and characterization of a H19 epitope point mutant of MDV CVI988/Rispens strain: Acta Virol. 43:169-173.

8. Cui, Z. Z., D. Yan, and L. F. Lee. 1990. Marek's disease virus gene clones encoding virus-specific phosphorylated polypeptides and serological characterization of fusion proteins. Virus Genes 3:309-322.

9. Davis, H. L. and M. J. McCluskie. 1999. DNA vaccines for viral diseases. Microbes. Infect. 1:7-21.

10. Davis, H. L., M. L. Michel, M. Mancini, M. Schleef, and R. G. Whalen. 1994. Direct gene transfer in skeletal muscle: plasmid DNA-based immunization against the hepatitis B virus surface antigen. Vaccine 12:1503-1509.

11. Delecluse, H. J. and W. Hammerschmidt. 1993. Status of Marek's disease virus in established lymphoma cell lines: herpes virus integration is common. J. Virol. 67:82-92.

12. Delecluse, H. J., S. Schuller, and W. Hammerschmidt. 1993. Latent Marek's disease virus can be activated from its chromosomally integrated state in herpes virus-transformed lymphoma cells. EMBO J. 12:3277-3286.

13. Fynan, E. F., R. G. Webster, D. H. Fuller, J. R. Haynes, J. C. Santoro, and H. L. Robinson. 1993. DNA vaccines: protective immunizations by parenteral, mucosal, and gene-gun inoculations. Proc. Natl. Acad. Sci. U.S.A 90:11478-11482.

14. Fynan, E. F., R. G. Webster, D. H. Fuller, J. R. Haynes, J. C. Santoro, and H. L. Robinson. 1995. DNA vaccines: a novel approach to immunization. Int. J. Immunopharmacol. 17:79-83.

15. Gimeno, I. M., R. L. Witter, and W. M. Reed. 1999. Four distinct neurologic syndromes in Marek's disease: effect of viral strain and pathotype. Avian Dis. 43:721-737.

16. Krishnan, B. R. 2000. Current status of DNA vaccines in veterinary medicine. Adv. Drug Deliv. Rev. 43:3-11.

17. Lee, L. F., P. Wu, D. Sui, D. Ren, J. Kamil, H. J. Kung, and R. L. Witter. 2000. The complete unique long sequence and the overall genomic organization of the GA strain of Marek's disease virus. Proc. Natl. Acad. Sci. U.S.A 97:6091-6096.

18. Messerle, M., I. Crnkovic, W. Hammerschmidt, H. Ziegler, and U. H. Koszinowski. 1997. Cloning and mutagenesis of a herpes virus genome as an infectious bacterial artificial chromosome. Proc. Natl. Acad. Sci. U.S.A. 94:14759-14763.

19. Morgan, R. W., J. L. Cantello, and C. H. McDermott. 1990. Transfection of chicken embryo fibroblasts with Marek's disease virus DNA. Avian Dis. 34:345-351.

20. Osterrieder, N., R. Wagner, C. Brandmuller, P. Schmidt, H. Wolf, and O. R. Kaaden. 1995. Protection against EHV-1 challenge infection in the murine model after vaccination with various formulations of recombinant glycoprotein gp14 (gB). Virology 208:500-510.

21. Rispens, B. H., H. van Vloten, N. Mastenbroek, H. J. Maas, and K. A. Schat. 1972. Control of Marek's disease in the Netherlands. I. Isolation of an avirulent Marek's disease virus (strain CVI988) and its use in laboratory vaccination trials. Avian Dis. 16:108-125.

22. Rispens, B. H., H. van Vloten, N. Mastenbroek, J. L. Maas, and K. A. Schat. 1972. Control of Marek's disease in the Netherlands. II. Field trials on vaccination with an avirulent strain (CVI988) of Marek's disease virus. Avian Dis. 16:126-138.

23. Robinson, H. L. and C. A. Torres. 1997. DNA vaccines. Semin. Immunol. 9:271-283.

24. Roy, K., H. Q. Mao, S. K. Huang, and K. W. Leong. 1999. Oral gene delivery with chitosan—DNA nanoparticles generates immunologic protection in a murine model of peanut allergy. Nat. Med. 5:387-391.

25. Saiki, R. K., T. L. Bugawan, G. T. Horn, K. B. Mullis, and H. A. Erlich. 1986. Analysis of enzymatically amplified beta-globin and HLA-DQ alpha DNA with allele-specific oligonucleotide probes. Nature 324:163-166.

26. Sambrook, J. and D. F. Fritsch and T. Maniatis. 1989. Molecular cloning: a laboratory manual. Cold Spring Harbor, N.Y.

27. SAS Institute Inc. 1984. SAS/STAT User's Guide, Version 6. SAS Institute Inc., Cary, N.C.

28. Schat, K. A., B. W. Calnek, and J. Fabricant. 1982. Characterization of two highly oncogenic strains of Marek's disease virus. Avian Pathol. 11:593-605.

29. Schumacher, D., B. K. Tischer, W. Fuchs, and N. Osterrieder. 2000. Reconstitution of Marek's disease virus serotype 1 (MDV-1) from DNA cloned as a bacterial artificial chromosome and characterization of a glycoprotein B-negative MDV-1 mutant. J. Virol. 74:11088-11098.

30. Sharma, J. M. 1971. In vitro cell association of Marek's disease herpes virus. Am. J. Vet. Res. 32:291-301.

31. Stokes, M. E., C. S. Davis, and G. G. Koch. 2000. Categorical Data Analysis Using the SAS System. SAS Institute Inc., Cary, N.C.

32. Suter, M., A. M. Lew, P. Grob, G. J. Adema, M. Ackermann, K. Shortman, and C. Fraefel. 1999. BAC-VAC, a novel generation of (DNA) vaccines: A bacterial artificial chromosome (BAC) containing a replication-competent, packaging-defective virus genome induces protective immunity against herpes simplex virus 1. Proc. Natl. Acad. Sci. U.S.A. 96:12697-12702.

33. Tulman, E. R., C. L. Afonso, Z. Lu, L. Zsak, D. L. Rock, and G. F. Kutish. 2000. The genome of a very virulent Marek's disease virus. J. Virol. 74:7980-7988.

34. Witter, R. L. 1982. Protection by attenuated and polyvalent vaccines against highly virulent strains of Marek's disease virus. Avian Pathol. 11:49-62.

35. Witter, R. L. 1983. Characteristics of Marek's disease viruses isolated from vaccinated commercial chicken flocks: association of viral pathotype with lymphoma frequency. Avian Dis. 27:113-132.

36. Witter, R. L. 1992. Safety and comparative efficacy of the CVI988/Rispens vaccine strain, p. 315-319. World's Poultry Science Assoc., Amsterdam.

37. Witter, R. L. 1997. Increased virulence of Marek's disease virus field isolates. Avian Dis. 41:149-163.

38. Witter, R. L. 2001. Protective efficacy of Marek's disease vaccines. Curr. Top. Microbiol. Immunol. 255:57-90.

39. Witter, R. L. and L. F. Lee. 1984. Polyvalent Marek's disease vaccines: safety, efficacy and protective synergism in chicken with maternal antibodies. Avian Pathol. 13:75-92.

40. Witter, R. L., L. F. Lee, and A. M. Fadly. 1995. Characteristics of CVI988/Rispens and R2/23, two prototype vaccine strains of serotype 1 Marek's disease virus. Avian Dis. 39:269-284.

41. Yokoyama, H., R. C. Peralta, S. Sendo, Y. Ikemori, and Y. Kodama. 1993. Detection of passage and absorption of chicken egg yolk immunoglobulins in the gastrointestinal tract of pigs by use of enzyme-linked immunosorbent assay and fluorescent antibody testing. Am. J. Vet. Res. 54:867-872.

TABLE 1
Testing of tumor formation in chickens after application of QM7 and SOgE cells

| | | Number of Animals with Tumors | | | | | | | | | | | | |
|---|---|---|---|---|---|---|---|---|---|---|---|---|---|---|
| | | Weeks post inoculation | | | | | | | | | | | | |
| Group | Animals (n) | 0 | 1 | 2 | 3 | 4 | 5 | 6 | 7 | 8 | 9 | 10 | 11 | Total |
| SOgE | 6 | | | | | | | | | | | | 0 | 0/6 |
| QM7 | 6 | | | | | | | | | | | | 0 | 0/6 |

TABLE 2
Marek's Disease in chickens immunized with vaccine strain CVI988 produced on
SOgE cells and challenged with hypervirulent strain EU1

| | | Number of Animals with Marek's Disease | | | | | | | | | | | |
|---|---|---|---|---|---|---|---|---|---|---|---|---|---|
| | Animals | Weeks after infection | | | | | | | | | | | |
| Group | (n) | 0 | 1 | 2 | 3 | 4 | 5 | 6 | 7 | 8 | 9 | 10 | Total |
| 1: SOgE | 7 | | | | | 1* | 1 | | 2 | | | 3 | 7/7 |
| 2: CVI988-SOgE | 8 | | | | | | | | | | | 1 | 1/8 |
| 3: CVI988-CEF | 8 | | | | | | | | | | | 1 | 1/8 |
| 4: QM7 | 6 | | | 2 | | 2 | 2 | | | | | | 6/6 |

*Numbers of animals given in weeks 0 to 9 died as a consequence of EU1 infection, numbers of chickens given in week 10 represent animals exhibiting MD at post mortem examination.

1. A process for producing a continuous cell line that supports the growth of a cell-associated alpha herpes virus, said process comprising:
infecting or transfecting a cell with a nucleic acid sequence of a herpes virus, and
culturing said infected or transfected cell or progeny thereof under conditions suitable for the expression of said nucleic acid sequence and propagation of said cell or progeny thereof,
thus producing a continuous cell line capable of supporting the growth of a cell-associated alpha herpes virus.

2. The process of claim 1 wherein said cell is an isolated vertebrate cell.

3. The process of claim 2 wherein said cell is an isolated avian cell.

4. The process of claim 1 wherein said cell is an isolated muscle myoblast cell.

5. The process of claim 4 wherein said muscle myoblast cell comprises QM7.

6. The process of claim 5 wherein said QM7 cell is as deposited at ATCC CRL-1632.

7. The process of claim 1 wherein said nucleic acid sequence comprises a glycoprotein gE gene of herpes virus.

8. The process of any one of claim 1 wherein said herpes virus is a Marek's disease virus.

9. The process of claim 8 wherein the Marek's disease virus is avirulent.

10. The process of claim 9 wherein said virus is Rispens CV1988.

11. The process of claim 1 wherein said continuous cell line is capable of supporting the growth of a Marek's disease virus strain without adaptation of said virus.

12. A cell produced by the process of claim 1 or a progeny of said cell.

13. A cell preparation produced by the cell of claim 12 or a progeny of said cell.

14. An improvement in a process for producing a vaccine capable of inducing protection against disease in a vertebrate of the type wherein a cell or cell preparation is used to produce the vaccine, the improvement comprising:
using the cell of claim 12 or a progeny of said cell, for preparing the vaccine.

15. The process of claim 14 wherein the vertebrate is avian.

16. A process of generating, maintaining, and/or isolating a Marek's disease virus strain, the process comprising:
using the cell or progeny thereof of claim 12 for the generation, maintenance, and/or isolation of the Marek's disease virus strain.

17. The process of claim 16 further comprising:
culturing the cell or progeny thereof with the Marek's disease virus in the presence of geneticin.

18. The process of claim 16 wherein the Marek's disease virus strain is a Marek's disease virus serotype 1.

19. The process of claim 16, wherein said Marek's disease virus strain is selected from the group consisting of EU1 , RB1B, 584Ap8OC, and CVI988.

20. The process of claim 16 wherein the Marek's disease virus strain comprises a natural or genetically modified strain.

21. An improvement in a process for producing an antigen in a cell, the improvement comprising:
using the cell of claim 12 for producing an antigen of a Marek's disease virus.

22. A process of generating and/or isolating and/or maintaining a Marek's disease virus strain, said process comprising:
infecting the cell of claim 12 or a progeny of said cell with said Marek's disease virus strain, and
culturing said cell under conditions suitable for the propagation of said cell and the generation, maintenance and isolation Marek's disease virus.

23. The process of claim 22 comprising wherein the cell is cultured with said virus in the presence of a compound selected from the group consisting of geneticin, a geneticin derivative, an analog of geneticin, or mixtures thereof.

24. The process of claim 22 or claim 23 wherein said Marek's disease virus strain is selected from the group consisting of a Marek's disease virus serotype 1, Marek's disease virus serotype 2, Marek's disease virus serotype 3, and mixtures of any thereof.

25. The process of claim 22, wherein said Marek's disease virus strain is EU1, RB1B, 584Ap8OC, or CVI988.

26. The process of claim 22 wherein said Marek's disease virus strain comprises a vaccine strain of Marek's disease virus.

27. A process for producing a diagnostic antigen of a Marek's disease virus, said process comprising:
providing the cell of claim 12 and
isolating components the cell,
thus producing a diagnostic antigen.

28. A process for producing a vaccine capable of inducing protection against disease in a vertebrate, said process comprising:
culturing the cell of claim 12,
harvesting cell culture components therefrom, and
using said cell culture components to produce said vaccine.

29. The process of claim 28 wherein said vertebrate is avian.

30. A vaccine obtainable by the process of claim 28.

31. A method of preventing, treating, or preventing and treating a herpes virus induced disease in a subject, the method comprising:
administering the vaccine of claim 30 to the subject.

32. The method according to claim 31 wherein the herpes virus induced disease is a Marek's disease virus associated disease.

33. The method according to claim 31 wherein said subject is avian.

34. A process for protecting a vertebrate against disease, said process comprising:
administering the vaccine of claim 30 which has been combined with a pharmaceutically acceptable carrier to a suitable vertebrate recipient.

35. The process of claim 34 wherein said vertebrate is avian.


SEQUENCE LISTING (4 sequences)

SEQ ID NO: 1 - DNA, 30 bp, Artificial Sequence
  Primer
cataagcatgcgagtcagcgtcataatgtg

SEQ ID NO: 2 - DNA, 30 bp, Artificial Sequence
  Primer
caagggcccatcagtggtataaatctaagc

SEQ ID NO: 3 - DNA, 20 bp, Artificial Sequence
  Forward primer
gcatatcagcctgttctatc

SEQ ID NO: 4 - DNA, 20 bp, Artificial Sequence
  Reverse primer
aaccaatggtcggctataac